(19) United States
(12) Patent Application Publication
(10) Pub. No.: US 2012/0264694 A1
(43) Pub. Date: Oct. 18, 2012

(54) COMPOSITION COMPRISING TORASEMIDE

(75) Inventors: Marinus Maria Martinus BOEREN, Oisterwijk (NL); Johanna Elisabeth Maria VERMEER, Middelbeers (NL); Henricus Franciscus Catharina MICHIELS, Reusel (NL)

(73) Assignee: EUROVET ANIMAL HEALTH B.V., Bladel (NL)

(21) Appl. No.: 13/445,380

(22) Filed: Apr. 12, 2012

Related U.S. Application Data
(60) Provisional application No. 61/510,548, filed on Jul. 22, 2011.

(30) Foreign Application Priority Data
Apr. 18, 2011 (NL) 2006622

(51) Int. Cl. A61K 31/44; A61K 31/501; A61K 31/55; A61P 11/08; A61K 31/7048; A61K 31/585; A61K 31/4422; A61P 9/00; C07D 213/74; A61K 38/05

(52) U.S. Cl. 514/16.3; 546/291; 514/346; 514/252.06; 514/212.07; 514/26; 514/171

(57) ABSTRACT

A composition for the alleviation or treatment of one or more symptoms caused by one or more conditions or diseases selected from the group consisting of dilated cardiomyopathy, acquired or naturally occurring valvular disease, hypertrophic cardiomyopathy, hypertrophic obstructive cardiomyopathy, restrictive cardiomyopathy and unclassified cardiomyopathy and combinations thereof. Also disclosed is a kit and a method for alleviating or treating the symptoms.

### PRIORITY CLAIM

This patent application claims priority to Dutch Patent Application No. 2006622, filed 18 Apr. 2011, and U.S. Provisional Patent Application No. 61/510,548, filed 22 Jul. 2011, the disclosures of which are incorporated herein by reference in their entirety.

### FIELD

Disclosed embodiments relate to a composition for the alleviation or treatment of one or more symptoms caused by one or more conditions or diseases. Disclosed embodiments also relate to a kit and a method for alleviating or treating the symptoms.

### BACKGROUND

Torasemide (also known as torsemide) is a loop diuretic and is, for example, disclosed in DE2516025 and U.S. Pat. No. 4,018,929. These documents also disclose the diuretic properties of torasemide in rats. Torasemide has the following formula:

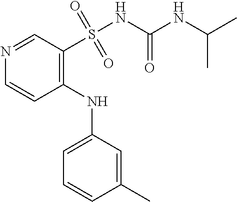

Further, diuretic properties in cats and dogs are disclosed by Ghys et al. (Arzneimittelforschung 1985;35 (10): 1520-6), Masami et al. (J. Vet. Med. Sci. 2003; 65 (10):1057-1061) and in dogs by Uchida et al. (J. Pharm. Pharmacol. 1992 January; 44(1):39-44). Matsuoka et al. discloses the use of torasemide in the treatment of congestive heart failure or edema in cats and dogs (J Vet Med Sci. 2003 October; 65 (10)1057-61).

Torasemide is further used in humans to treat edema associated with congestive heart failure or to treat high blood pressure. Further uses of torasemide in the treatment of heart failure, cirrhosis, pulmonary hypertension, chronic kidney disease and acute kidney disease in humans are reviewed by Wargo and Banta in The Annals of Pharmacotherapy, 2009 November, Volume 43.

None of the documents disclose the use of torasemide in the alleviation or treatment of one or more symptoms caused by one or more conditions or diseases selected from the group consisting of dilated cardiomyopathy, acquired or naturally occurring valvular disease, hypertrophic cardiomyopathy, hypertrophic obstructive cardiomyopathy, restrictive cardiomyopathy and unclassified cardiomyopathy and combinations thereof. Patients suffering from these diseases can suffer greatly from symptoms caused by one or more of these diseases. Up until now it was not recognized that torasemide can be used in the treatment or alleviation of certain symptoms, more specifically symptoms caused by one or more conditions or diseases selected from the group consisting of dilated cardiomyopathy, acquired or naturally occurring valvular disease, hypertrophic cardiomyopathy, hypertrophic obstructive cardiomyopathy, restrictive cardiomyopathy and unclassified cardiomyopathy and combinations thereof.

### SUMMARY

Disclosed embodiments provide for the alleviation or treatment of one or more symptoms caused by one or more conditions or diseases selected from the group consisting of dilated cardiomyopathy, acquired or naturally occurring valvular disease, hypertrophic cardiomyopathy, hypertrophic obstructive cardiomyopathy, restrictive cardiomyopathy and unclassified cardiomyopathy and combinations thereof

### DETAILED DESCRIPTIONS OF THE DISCLOSED EMBODIMENTS

At least one disclosed embodiment provides for the use of torasemide in the alleviation or treatment of symptoms caused by one or more conditions or diseases selected from the group consisting of dilated cardiomyopathy, acquired or naturally occurring valvular disease, hypertrophic cardiomyopathy, hypertrophic obstructive cardiomyopathy, restrictive cardiomyopathy and unclassified cardiomyopathy and combinations thereof.

Torasemide is available on the market as Torasemide by Cosma SpA IT 24040 Ciserano (Bergamo) and by S.I.M.S. S.R.L. IT 50066 Reggello. Torasemide has several advantages, for example, it has a longer duration of action, a higher potency, a stronger effect of action, less side effects and the patient develops no resistance if compared to current diuretics like furosemide.

The term patient(s) is meant to include one or more mammals and may be a dog and/or cat suffering from dilated cardiomyopathy, acquired or naturally occurring valvular disease, hypertrophic cardiomyopathy, hypertrophic obstructive cardiomyopathy, restrictive cardiomyopathy and unclassified cardiomyopathy or combinations thereof.

The use of a composition comprising torasemide as a veterinary medicament can alleviate or treat symptoms caused by one or more conditions or diseases, selected from the group consisting of dilated cardiomyopathy, acquired or naturally occurring valvular disease, hypertrophic cardiomyopathy, hypertrophic obstructive cardiomyopathy, restrictive cardiomyopathy and unclassified cardiomyopathy and combinations thereof This greatly reduces the suffering of patients and hence improves the quality of life of the patient.

If the disease is one or more chosen from the group consisting of dilated cardiomyopathy, acquired or naturally occurring valvular disease, and unclassified cardiomyopathies and combinations thereof, the use of torasemide in the alleviation or treatment of symptoms caused by the diseases is especially effective. Hence the suffering of patients caused by the diseases is reduced and the quality of life of the patient improved.

In at least one disclosed embodiment, the composition further comprises at least one additional medicament, selected from the group, consisting of pimobendan (a benzimidazole-pyridazinone derivative), one or more ACE inhibitors (e.g. benazepril, enalapril and ramipril), one or more β-blockers (e.g. propanolol, atenolol), one or more cardiac glycosides (e.g. digoxin), one or more aldosteron antagonists (e.g. spironolactone), one or more calcium channel blockers (e.g. amlodipine), one or more bronchodilatators and combinations thereof.

In at least one disclosed embodiment, the composition is provided to a subject in combination with a separate dose of at least one additional medicament, selected from the group, consisting of pimobendan (a benzimidazole-pyridazinone derivative), one or more ACE inhibitors (e.g. benazepril, enalapril and ramipril), one or more β-blockers (e.g. propanolol, atenolol), one or more cardiac glycosides (e.g. digoxin), one or more aldosteron antagonists (e.g. spironolactone), one or more calcium channel blockers (e.g. amlodipine), one or more bronchodilatators and combinations thereof.

In another disclosed embodiment, the veterinary medicament is for cats and/or dogs.

In yet another disclosed embodiment, the veterinary medicament is for a cat and the additional medicament is selected from the group, consisting of pimobendan (a benzimidazole-pyridazinone derivative), one or more ACE inhibitors (e.g. benazepril, enalapril and ramipril), one or more β-blockers (e.g. propanolol, atenolol), one or more cardiac glycosides (e.g. digoxin), one or more aldosteron antagonists (e.g. spironolactone), one or more calcium channel blockers (e.g. amlodipine), one or more bronchodilatators and combinations thereof.

In a further disclosed embodiment, the veterinary medicament is for a dog and the composition comprises or is provided to a subject in combination with pimobendan.

Optionally, one or more of the crystalline forms of pimobendan as described in NL1037569 is used. Suitable daily dosages of pimobendan are maximally 0.6 mg/kg body weight of the subject. The solubility characteristics of the crystalline forms are such that adding an organic acid or an anhydride thereof is not needed for ensuring a satisfactory dissolution rate and therefore satisfactory resorption of pimobendan.

In at least one disclosed embodiment, the veterinary medicament is for the alleviation or treatment of symptoms caused by a condition or disease selected from the group consisting of dilated cardiomyopathy, acquired or naturally occurring valvular disease, or unclassified cardiomyopathies and where an augmentation of cardiac output is possible.

In another disclosed embodiment, the medicament is provided to the subject in a dosage of at the most 5 mg/kg body weight of the subject, and optionally 0.025 to 0.29 mg/kg body weight of the subject or a dosage of 0.31 to 5 mg/kg body weight of the subject. The dosage may be administered once daily. Within these ranges, the present inventors observed a good effect.

In at least one disclosed embodiment, the composition is administered orally, sublingually, intramuscularly, subcutaneously, intravenously or transdermally.

In another disclosed embodiment, the composition is a formulation for parenteral administration and may comprise torasemide, preservative(s), isotonic agent(s), buffering agent(s) and solvent(s). Optionally torasemide is present as anhydrous torasemide.

In yet another disclosed embodiment, the composition is in tablet form and comprises torasemide, one or more diluent(s) and/or filler(s), disintegrant(s), lubricant(s) and colorant(s) and optionally one or more flavourants. Optionally torasemide is present as anhydrous torasemide.

In still another disclosed embodiment, the composition is a palatable composition. The acceptance of the composition by the subjects is improved and force-feeding of subjects can be avoided with the palatable composition. This leads to a high compliance of the subjects to be treated.

Another disclosed embodiment also relate to a kit comprising a composition comprising torasemide and further comprising a package leaflet or user instruction including information for use.

Yet another disclosed embodiment relates to a method for alleviating or treating symptoms in an animal caused by one or more conditions or diseases, selected from the group consisting of dilated cardiomyopathy, acquired or naturally occurring valvular disease, hypertrophic cardiomyopathy, hypertrophic obstructive cardiomyopathy, restrictive cardiomyopathy and unclassified cardiomyopathy and combinations thereof comprising the step of administering to the animal a composition comprising torasemide.

### EXAMPLES

As an example for the use of the composition, a formulation for parenteral administration comprises the following:
Torasemide (anhydrous)
Preservative(s)
Isotonic agent(s)
Buffering agent(s)
Solvent(s)

As an example for the use of the composition, a formulation for a tablet for oral administration comprises the following ingredients:
Torasemide (anhydrous)
Diluent(s)/Filler(s)
Disintegrant(s)
Lubricant(s)
Colorant(s)
Flavour(s)

1. A Composition comprising torasemide for use as a veterinary medicament for the alleviation or treatment of symptoms caused by one or more conditions or diseases, selected from the group consisting of dilated cardiomyopathy, acquired or naturally occurring valvular disease, hypertrophic cardiomyopathy, hypertrophic obstructive cardiomyopathy, restrictive cardiomyopathy and unclassified cardiomyopathy and combinations thereof.

2. The composition of claim 1, wherein the disease is one or more selected from the group consisting of dilated cardiomyopathy, acquired or naturally occurring valvular disease, and unclassified cardiomyopathies and combinations thereof.

3. The composition of claim 1, wherein the composition further comprises at least one additional medicament selected from the group consisting of pimobendan (a benzimidazole-pyridazinone derivative), one or more ACE inhibitors (e.g. benazepril, enalapril and ramipril), one or more β-blockers (e.g. propanolol, atenolol), one or more cardiac glycosides (e.g. digoxin), one or more aldosteron antagonists (e.g. spironolactone), one or more calcium channel blockers (e.g. amlodipine), one or more bronchodilatators and combinations thereof.

4. The composition of claim 1, wherein the composition is provided to a subject in combination with a separate dose of at least one additional medicament selected from the group consisting of pimobendan (a benzimidazole-pyridazinone derivative), one or more ACE inhibitors (e.g. benazepril, enalapril and ramipril), one or more β-blockers (e.g. propanolol, atenolol), one or more cardiac glycosides (e.g. digoxin), one or more aldosteron antagonists (e.g. spironolactone), one or more calcium channel blockers (e.g. amlodipine), one or more bronchodilatators and combinations thereof.

5. The composition of claim 1, wherein the veterinary medicament is for mammals.

6. The composition of claim 5, wherein the veterinary medicament is for a cat and the additional medicament is selected from the group, consisting of pimobendan, one or more ACE inhibitors (e.g. benazepril, enalapril and ramipril), one or more β-blockers (e.g. propanolol, atenolol), one or more cardiac glycosides (e.g. digoxin), one or more aldosteron antagonists (e.g. spironolactone), one or more calcium channel blockers (e.g. amlodipine), one or more bronchodilatators and combinations thereof.

7. The composition of claim 3, wherein the veterinary medicament is for a cat and/or a dog and the composition comprises pimobendan or is provided to a subject in combination with pimobendan.

8. The composition of claim 5, wherein the veterinary medicament is for the alleviation or treatment of symptoms caused by a condition or disease selected from the group consisting of dilated cardiomyopathy, acquired or naturally occurring valvular disease, or unclassified cardiomyopathies and where an augmentation of cardiac output is possible.

9. The composition of claim 1, wherein the medicament is provided to the subject in a dosage of 0.025 to 0.29 mg/kg or a dosage of 0.31 to 5 mg/kg once daily.

10. The composition of claim 1, wherein the composition is a palatable composition.

11. A kit comprising a composition according to claim 1 and further comprising a package leaflet or user instruction including information for the use according to claim 1.

12. A method for alleviating or treating symptoms in an animal caused by one or more conditions or diseases selected from the group consisting of dilated cardiomyopathy, acquired or naturally occurring valvular disease, hypertrophic cardiomyopathy, hypertrophic obstructive cardiomyopathy, restrictive cardiomyopathy and unclassified cardiomyopathy and combinations thereof comprising the step of administering to the animal a composition comprising torasemide.
